(19) United States
(12) Patent Application Publication
(10) Pub. No.: US 2002/0028772 A1
(43) Pub. Date: Mar. 7, 2002

(54) Modulators of activity of G-protein-coupled receptor kinases

(75) Inventor: Shmuel A. Ben-Sasson, Jerusalem (IL)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Children's Med. Corporation,

(21) Appl. No.: 09/735,274

(22) Filed: Dec. 11, 2000

Related U.S. Application Data
(63) Continuation-in-part of application No. PCT/US98/10319, filed on May 20, 1998.
Application No. PCT/US98/10319 is a continuation-in-part of application No. 08/861,338, filed on May 21, 1997.

(51) Int. Cl. A61K 38/17

(52) U.S. Cl. 514/12

(57) ABSTRACT

Disclosed is a method for modulating metabolism in an individual. The method includes administering to the individual a substance, such as a GRK-derived HJ loop peptide, which alters activity of a GRK, wherein the administration of the substance results in an increase or decrease of the individual's metabolism. Also disclosed are GRK-derived HJ loop peptides.

### RELATED APPLICATIONS

This application is a continuation-in-part of International Application No. PCT/US98/10319 filed May 20, 1998, which is a continuation-in-part of U.S. application Ser. No. 08/861,338, filed May 21, 1997. The entire teachings of the above applications are incorporated herein by reference.

### BACKGROUND OF THE INVENTION

Serine/threonine kinases are a member of the eukaryotic protein kinase superfamily. Enzymes of this class specifically phosphorylate serine or threonine residues of intracellular proteins and are important in mediating signal transduction in multicellular organisms. Many serine/threonine kinases occur as intracellular proteins which take part in signal transduction within the cell, including signal transduction to the nucleus and the activation of other proteins. A particular group of serine/threonine kinases, G protein-coupled receptor kinases, are found in cell membranes and participate in trans-membrane signalling.

As such, phosphorylation of serine or threonine by serine/threonine kinases is an important mechanism for regulating intracellular events in response to environmental changes. A wide variety of cellular events are regulated by serine/threonine kinases. A few examples include the ability of cells to enter and/or complete mitosis, cellular proliferation, cellular differentiation, the control of fat metabolism, immune responses, inflammatory responses and the control of glycogen metabolism.

An important superfamily of cell membrane receptors is the group known as G-protein coupled receptors (GPCR), known also as seven trans-membrane receptors (7TM). This superfamily of receptors is involved in the transmission of signals that originate from low molecular weight ligands such as adrenaline or from peptide ligands such as chemokines and a variety of hormones such as melanocyte stimulating hormone (MSH).

Numerous studies have shown that intracellular protein kinases which specifically interact with various members of the 7TM receptors are able to desensitize them and thereby weaken the signal or prevent it from being effected. These protein kinases are known as G-protein-coupled receptor kinases (GRKs). So far, six of these kinases have been discovered (GRK1-6). Some of the GRKs are restricted to a small number of tissues (e.g., GRK-1), while GRK2 and 3, known also as βARK1 and 2 are ubiquitously expressed. A comprehensive review is provided, for example, by M. Bunemann and M. M. Hosey, “G-Protein Coupled Receptor Kinases as Modulators of G-Protein Signalling,” J. of Physiology, Vol. 517(1):5-23 (1999).

It is, therefore, important to find methods and substances to modulate (increase or decrease) the function of 7TM receptors, perhaps via the modulation of GRK activity. Such modulation could be used to treat a wide variety of diseases and conditions associated with 7TM receptor function.

### SUMMARY OF THE INVENTION

In one aspect, the invention relates to methods of modulating (increasing or inhibiting) metabolism in an individual. The methods include administration of a substance which alters G-protein-coupled receptor kinase 2 (GRK2) activity or G-protein-coupled receptor kinase 3 (GRK3) activity in the individual, wherein the administration results in an increase or a decrease of metabolism. Examples of metabolism modulation include, but are not limited to, enhancing melanogenesis, altering syndrome X, correcting Type II diabetes mellitus, relieving hypertension, improving heart function and lowering propensity towards obesity. In a preferred embodiment, the activity of the GRK2 or GRK3 is altered as the kinase interacts with a seven trans-membrane receptor (7TM) and thus affects or interferes with the receptor's ability to carry out its function when its complementary ligand is present. For instance, inhibition of the GRK2 or GRK3 activity results in increased activity of the interacting 7TM receptor as it carries out its function in the presence of its complementary ligand. In a preferred embodiment, the substance which alters the activity of GRK2 or GRK3 is a peptide, such as a GRK-derived HJ loop peptide.

In another aspect, the invention relates to a method of modulating activity of a GRK in a laboratory animal or an individual suffering from a medical indication such as, for example, Type II diabetes mellitus, obesity or syndrome X. The method includes administering to the animal or individual a GRK-derived HJ loop peptide.

In a further aspect, the invention relates to a method of modulating site specific activity of a G-protein coupled receptor kinase (GRK) in a laboratory animal or an individual in need of enhanced or reduced signaling of a seven trans-membrane receptor. The method includes administering site specifically a GRK-derived HJ loop peptide, thereby modulating the site specific activity of the GRK. For example, a method of inhibiting site specific activity of a G-protein coupled receptor kinase (GRK) includes administering site specifically, a GRK-derived HJ loop peptide, thereby inhibiting the site specific activity of the GRK.

The invention also relates to GRK-derived HJ loop peptides, in particular GRK2-and GRK3-derived HJ loop peptides. Particularly preferred are peptides K024H001 (SEQ ID NO.:1), K024H003 (SEQ ID NO.:2), K024H007 (SEQ ID NO.:3), K024H101 (SEQ ID NO.:4), K024H102 (SEQ ID NO.:5), K024H103 (SEQ ID NO.:6), K024H104 (SEQ ID NO.:7), K024H105 (SEQ ID NO.:8), K024H106 (SEQ ID NO.:9), K024H107 (SEQ ID NO.:10), K024H108 (SEQ ID NO.:11), K024H109 (SEQ ID NO.:12), K024H10 (SEQ ID NO.:13), K024H111 (SEQ ID NO.:14), K024H112 (SEQ ID NO.:15), K024H113 (SEQ ID NO.:16), K024H114 (SEQ ID NO.:17), K024H901 (SEQ ID NO.:18), and K024H903 (SEQ ID NO.:19).

The invention further relates to methods of treating syndrome X or Type II diabetes mellitus in individuals by administering one or more inhibitors of GRK2 or GRK3 to these individuals. The administration of inhibitors GRK2 or GRK3 causes an improvement in syndrome X and corrects Type II diabetes mellitus in those individuals.

Any inhibitor of GRK2 or GRK3 can be administered to the individuals in the course of treating syndrome X or Type II diabetes mellitus. Among the GRK2 or GRK3 inhibitors that can be employed are peptides, antibodies immunoreactive with GRK2 or GRK3, anti-sense nucleic acids that block expression of GRK2 or GRK3, negative dominant GRK2 or GRK3 genes which express GRK2 or GRK3 proteins with reduced or non-existent biological activity, and small organic molecules. Any of these inhibitors of GRK2 or GRK3 will correct Syndrome X and Type II diabetes mellitus.

The invention has many advantages. For example, the invention provides methods of modulating metabolism in an individual and thus is useful in treating indications such as Type II diabetes, propensity for obesity, syndrome X and others. The invention also provides methods for modulating the local activity of a GRK. Furthermore, the invention discloses short peptides which are capable of modulating activity of GRKs. These peptides can be manufactured cost effectively and are easy to administer.

### BRIEF DESCRIPTION OF THE DRAWINGS

FIG. 1 is a Table illustrating the amino acid sequences of the HJ loop of GRK2 and GRK3, also referred to herein as βARK1 (or βARK1) and βARK2 (or PARK2).

FIG. 2 is a Table illustrating the sequences of peptides K024H001 (SEQ ID NO.:1), K024H003 (SEQ ID NO.:2), K024H007 (SEQ ID NO.:3), K024H101 (SEQ ID NO.:4), K024H102 (SEQ ID NO.:5), K024H103 (SEQ ID NO.:6), K024H104 (SEQ ID NO.:7), K024H105 (SEQ ID NO.:8), K024H106 (SEQ ID NO.:9), K024H107 (SEQ ID NO.:10), K024H108 (SEQ ID NO.:11), K024H109 (SEQ ID NO.:12), K024H110 (SEQ ID NO.:13), K024H111 (SEQ ID NO.:14), K024H112 (SEQ ID NO.:15), K024H113 (SEQ ID NO.:16), K024H114 (SEQ ID NO.:17), K024H901 (SEQ ID NO.:18), and K024H903 (SEQ ID NO.:19).

FIG. 3 is a graph that shows the effects of a single injection of the GRK-derived peptide on blood-glucose of sand rats (psamomys obesus).

FIG. 4 is a graph that shows blood-glucose of control sand rats (psamomys obesus).

FIGS. 5A-5F are graphs illustrating the effects of peptides of the invention on melanogenesis by murine B 16 melanoma cells.

### DETAILED DESCRIPTION OF THE INVENTION

A serine/threonine kinase (hereinafter “STK”) is an intracellular or membrane bound protein which uses the gamma phosphate of ATP or GTP to generate phosphate monoesters on the hydroxyl group of a serine or threonine residue. STKs have homologous “kinase domains” or “catalytic domains” which carry out this phosphorylation. Based on a comparison of a large number of protein kinases, it is now known that the kinase domain of protein kinases, including STKs, can be divided into twelve subdomains, which are regions generally uninterrupted by large amino acid insertions and contain characteristic patterns of conserved residues (Hanks and Hunter, “The Eukaryotic Protein Kinase Superfamily”, in Hardie and Hanks (ed.), The Protein Kinase Facts Book, Volume I, Academic Press, Chapter 2, 1995. These subdomains are referred to as Subdomain I through Subdomain XII.

The “HJ loop” referred to herein is found within the kinase domain of STKs between the middle of Subdomain IX and the middle of Subdomain X. Because of the high degree of homology found in the subdomains of different protein kinases, including STKs, the amino acid sequences of the domains of different STKs can be aligned. Thus, the HJ loop of a STK can be defined by reference to the amino acid sequence of a prototypical protein kinase, for example PKA-Cα, and can be said to correspond to a contiguous sequence of about twenty amino acid residues found between about amino acid 229 and 248 of PKA-Cα.

A second definition of the HJ loop of a STK, which is complementary to the definition provided in the proceeding paragraph, can be made by reference to the three dimensional structure of the kinase domain of STKs. The kinase domain of STKs has been found to contain at least nine alpha helices, referred to as helix A through helix I (Tabor et al., Phil. Trans. R. Soc. Lond. B340:315 (1993), Mohammadi et al., Cell 86:577 (1996) and Hubbard et al, Nature 372:746 (1994)). The HJ loop is a contiguous sequence of about twenty amino acids beginning within the F helix about five amino acids residues from the N-terminus of the F helix and extending about five amino acid residues into the G helix.

A wide variety of cellular events are regulated by serine/threonine kinases. A few examples include the ability of cells to enter and/or complete mitosis, cellular proliferation, cellular differentiation, the control of fat metabolism, immune responses, inflammatory responses and the control of glycogen metabolism.

Among serine/threonine kinases, G-protein coupled receptor kinases (GRKs) have been implicated in the regulation of various hormonal responses, as discussed, for example, by Freedman and Lefkowitz, Recent Prog. Hormon. Res. 51:319 (1996). It has been reported that GRKs specifically interact with various members of the 7TM receptors.

It has been found with the present invention that modulation of activity of GRKs influences a variety of signal-transduction pathways. For example, inhibition of a GRK can result in a stronger or more extended signal by its corresponding 7TM receptor; e.g., extending the duration of hormonal effects of, for example, adrenaline. Thus, agents which modulate the activity of G-protein receptor kinase can be used in the treatment of diseases that result from a lower bioavailability of the corresponding ligand, such as dopamine.

A particular intriguing situation with this invention is the systemic administration of a GRK 2,3 inhibitor. Under such circumstances, multiple systems can be affected simultaneously. Without wishing to be bound by a particular mechanism, it is believed that, if all of the systems which control the metabolic activity of the body are tuned by the same molecular mechanism, namely GRK activity, then a systemic inhibition of GRK 2, 3 will have a simple phenotypic result: increase in the overall body basal metabolic rate. Such a result is favorable in the condition now known as “syndrome-x” which is typified by the onset of type II diabetes mellitus, obesity and other conditions. For a review of syndrome-x, see O. Timar et al, “Metabolic Syndrome X: A Review,” Can. J. Cardiol, 16(6): 779-789 (2000).

With this invention, inhibiting the effects of GRK-2, which can be thought of as a metabolic regulator, is a method of treating type 2 diabetes mellitus (DM). It appears that in specific low calorie environments, organisms including humans, have evolved a mechanism by which maximal energy metabolism is achieved by down-regulating metabolic processes. It is postulated in this invention that the mechanism for this down-regulation is phosphorylation of β-adrenergic receptors (βAR) by GRK-2 (β-adrenergic receptor kinase). The attenuated βAR leads to decreased signaling to significant metabolic processes such as glucose uptake (via insulin resistance), lipid breakdown, etc. This enables the organism to maintain energy homeostasis despite low exogenous caloric intake.

Nutritional diabetes can be caused by a pathologic function of the interaction between βAR and GRK-2. When organisms that are maximally adapted to a low energy environment are transferred to a high-energy environment, they develop a metabolic syndrome characterized by type 2 diabetes mellitus (DM), hypertension, obesity, insulin resistance, etc. This is due to the surfeit of energy, which is inefficiently utilized because of the low metabolic rate. The surplus energy is converted to fat and there is a hyperglycemia due to insulin resistance in the face of high glucose levels. By decreasing the activity of GRK-2, the activity of βAR is increased and the metabolic rate is increased.

The concept of a metabolic regulator comes from an animal model of nutritional DM. Psamomys obesus, a desert gerbil that survives on a low energy diet, develops insulin resistance and type 2 DM when placed on a high energy diet. As shown herein, diabetes is corrected when GRK-2 activity is inhibited, thereby supporting the concept that manipulation of a metabolic rheostat is a treatment for DM.

The following information further substantiates the concept of such a metabolic rheostat: Upregulation of GRK also causes decreased βAR in the heart which exacerbates heart failure. Inhibition of GRK by a peptide inhibitor delivered locally to the heart improves function. High GRK-2 levels are associated with hypertension. GRK-2 has a role in insulin secretion. GRK has a role in CNS signaling. GRK has a role in hormone secretion. GRK has a role in olfaction.

In one embodiment, the invention is directed to a method of modulating metabolism in a subject or individual. The method includes the administration of a substance which alters G-protein-coupled receptor kinase 2 (GRK2) activity or G-protein-coupled receptor kinase 3 (GRK3) activity in the subject or individual, wherein the administration results in an increase or a decrease of metabolism. In a preferred embodiment, GRK2 or GRK3 interacts with a 7TM receptor, thereby affecting the ability of the receptor to carry out its function when its complementary ligand is present and the activity of the GRK is altered. Complementary ligands of 7TM receptors whose activity is modulated by GRKs are known in the art. Examples of complementary ligands of 7TM receptors modulated by GRK2 and GRK3 include, but are not limited to adrenaline, angiotensin, chemokines, dopamine, acetyl-choline and opioids.

A “subject” is preferably a human, but can also be animals in need of treatment, e.g., veterinary animals (e.g., dogs, cats, and the like), farm animals (e.g., cows, pigs, horses, chickens and the like) and laboratory animals (e.g., rats, mice, guinea pigs and the like).

As used herein, the term “modulating”, used interchangeably with altering or changing refers to an increase or decrease, in a biological, physiological or cellular function or activity, as compared to controlled conditions. Modulation of an individual's metabolism refers to an inhibition or enhancement of metabolic processes such as glucose uptake (via insulin resistance), lipid breakdown or synthesis, gluconeogenesis, glycogenolysis, cellular uptake of free fatty acids and triglycerides and cholesterol metabolism compared to a base line level for the individual, as known in the art.

In a preferred embodiment, “modulated metabolism” refers to enhanced melanogenesis, alteration of syndrome X, corrected Type II diabetes mellitus, improvement of heart function, relief of hypertension and lowered propensity for obesity. Methods of determining changes in these functions and activities are well known in the art and are further described below.

Preferred substances which can be used in the methods of the invention, include GRK-derived HJ loop peptides, i.e. peptides and peptide derivatives from the HJ loop of GRK serine/threonine kinases.

Optionally, the C-terminus or the N-terminus of the peptides of the present invention, or both, can be substituted with a carboxylic acid protecting group or an amine protecting group, respectively. Suitable protecting groups are described in Green and Wuts, “Protecting Groups in Organic Synthesis”, John Wiley and Sons, Chapters 5 and 7, 1991, the teachings of which are incorporated herein by reference. Preferred protecting groups are those that facilitate transport of the peptide into a cell, for example, by reducing the hydrophilicity and increasing the lipophilicity of the peptide. In addition, a modified lysine residue can be added to the carboxy terminus to enhance biological activity. Examples of the lysine modification include the addition of an aromatic substitute, such as benzoyl, or an aliphatic group, such as acyl, or a myristic or stearic acid, at the epsilon amino group of the lysine residue. Examples of N-terminal protecting groups include acyl groups (—CO—R₁) and alkoxy carbonyl or aryloxy carbonyl groups (—CO—O—R₁), wherein R₁ is an aliphatic, substituted aliphatic, benzyl, substituted benzyl, aromatic or a substituted aromatic group. Specific examples of acyl groups include acetyl, (ethyl)-CO—, n-propyl-CO—, iso-propyl-CO—, n-butyl-CO—, sec-butyl-CO—, t-butyl-CO—, hexyl, lauroyl, palmitoyl, myristoyl, stearyl, phenyl-CO—, substituted phenyl-CO—, benzyl-CO— and (substituted benzyl)-CO—. Examples of alkoxy carbonyl and aryloxy carbonyl groups include CH₃-O—CO—, (ethyl)-O—CO—, n-propyl-O—CO—, iso -propyl-O—CO—, n-butyl-O—CO—, sec-butyl-O—CO—, t-butyl-O—CO—, phenyl-O—CO—, substituted phenyl-O—CO—and benzyl-O—CO—, (substituted benzyl)-O—CO—. In order to facilitate the N-acylation, one to four glycine residues can be added to the N-terminus of the sequence. The carboxyl group at the C-terminus can be protected, for example, by an amide (i.e., the hydroxyl group at the C-terminus is replaced with —NH₂, —NHR₂ and—NR₂R₃) or ester (i.e. the hydroxyl group at the C-terminus is replaced with —OR₂). R₂ and R₃ are independently an aliphatic, substituted aliphatic, benzyl, substituted benzyl, aryl or a substituted aryl group. In addition, taken together with the nitrogen atom, R₂ and R₃ can form a C4 to C8 heterocyclic ring with from about 0-2 additional heteroatoms such as nitrogen, oxygen or sulfur. Examples of suitable heterocyclic rings include piperidinyl, pyrrolidinyl, morpholino, thiomorpholino or piperazinyl. Examples of C-terminal protecting groups include —NH₂, —NHCH₃, —N(CH₃)₂, —NH(ethyl),—N(ethyl)₂, —N(methyl)(ethyl), —NH(benzyl), —N(C1-C4 alkyl)(benzyl), —NH(phenyl),—N(C1-C4 alkyl)(phenyl), —OCH₃, —O—(ethyl), —O—(n-propyl), —O—(n-butyl), —O—(iso-propyl), —O—(sec-butyl), —O—(t-butyl), —O—benzyl and —O—phenyl.

An “amino acid residue” is a moiety found within a peptide and is represented by —NH—CHR—CO—, wherein R is the side chain of a naturally occurring amino acid. When referring to a moiety found within a peptide, the terms “amino acid residue” and “amino acid” are used in this application. An “amino acid residue analog” is either a peptidomimetic or is a D or L residue having the following formula: —NH—CHR—CO—, wherein R is an aliphatic group, a substituted aliphatic group, a benzyl group, a substituted benzyl group, an aromatic group or a substituted aromatic group and wherein R does not correspond to the side chain of a naturally-occurring amino acid. When referring to a moiety found within a peptide, the terms “amino acid residue analog” and “amino acid analog” are used interchangeably in this application. Amino acid analogs are well-known in the art; a large number of these analogs are commercially available.

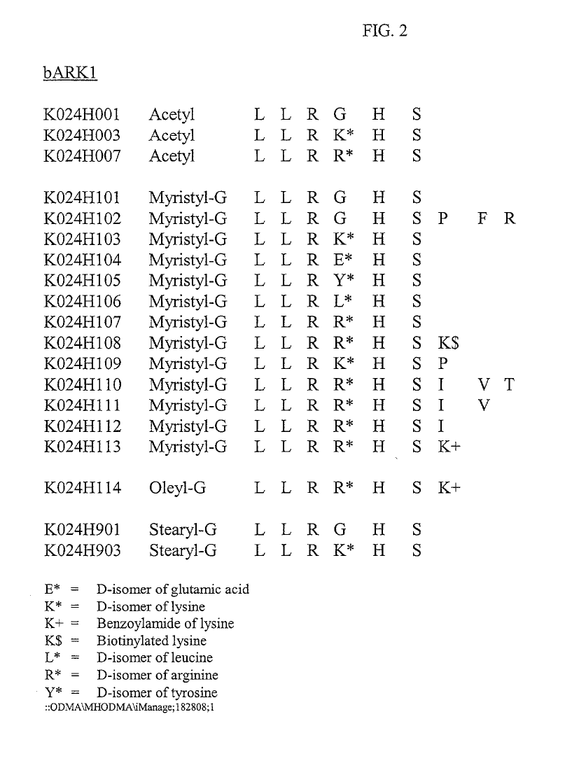

A “conservatively substituted amino acid”, also called a “conservatively substituted amino acid residue”, is an amino acid analog which, when substituted for a native (original) amino acid of the HJ loop peptides (shown herein as the peptides with SEQ ID numbers of FIG. 2 but without the N-terminal glycines or the denoted amino acid substitutions of these sequences) or is inserted as a spacer group in the amino acid sequence of the HJ loop peptides, does not severely alter the modulating activity of the peptide. A peptidomimetic of the naturally occurring amino acid, as well documented in the literature known to the skilled practitioner, also referred to as a “functional peptidomimetic” is an organic moiety which, when substituted for a native (original) amino acid of the HJ loop peptides or is inserted as a spacer group in the amino acid sequence of the HJ loop peptides, also does not severely alter the modulating activity of the peptide. The ability of such a HJ loop peptide derivative to affect the activities of 7TM receptors via the interaction of the peptide derivative with a GRK is not markedly different from the modulating ability of the native or original HJ loop peptide either when a conservatively substituted amino acid analog or functional peptidomimetic replaces a native amino acid of the native or original HJ loop peptide, or when a conservatively substituted amino acid analog or functional peptidomimetic is inserted in an amino acid sequence of a HJ loop peptide. Since such HJ loop peptide derivatives have modulating ability which is essentially the same as that of HJ loop peptides, these HJ loop peptide derivatives are also embodiments of this invention.

As used herein, aliphatic groups are straight chained, branched or cyclic C1-C8 hydrocarbons that are completely saturated, which contain one or two heteroatoms such as nitrogen, oxygen or sulfur and/or which contain one or more units of unsaturation. Aromatic groups are carbocyclic aromatic groups such as phenyl and naphthyl and heterocyclic aromatic groups such as imidazolyl, indolyl, thienyl, furanyl, pyridyl, pyranyl, pyrrolyl, oxazolyl, benzothienyl, benzofuranyl, quinolinyl, isoquinolinyl and acridintyl.

Suitable substituents on an aliphatic, aromatic or benzyl group include —OH, halogen (—Br, —Cl, —I and —F), —O(aliphatic, substituted aliphatic, benzyl, substituted benzyl, aryl or substituted aryl group), —CN, —NO₂, —COOH, —NH2, —NH(aliphatic group, substituted aliphatic, benzyl, substituted benzyl, aryl or substituted aryl group), —N(aliphatic group, substituted aliphatic, benzyl, substituted benzyl, aryl or substituted aryl group)₂, —COO(aliphatic group, substituted aliphatic, benzyl, substituted benzyl, aryl or substituted aryl group), —CONH₂, —CONH(aliphatic, substituted aliphatic group, benzyl, substituted benzyl, aryl or substituted aryl group), —SH, —S(aliphatic, substituted aliphatic, benzyl, substituted benzyl, aromatic or substituted aromatic group) and —NHC—C(═NH)—NH₂. A substituted benzylic or aromatic group can also have an aliphatic or substituted aliphatic group as a substituent. A substituted aliphatic group can also have a benzyl, substituted benzyl, aryl or substituted aryl group as a substituent. A substituted aliphatic, substituted aromatic or substituted benzyl group can have one or more of these substituents.

Suitable substitutions for amino acid residues in the sequence of a HJ loop peptide include conservative substitutions which result in peptide derivatives which modulate the activity of a GRK. Among the categories of amino acid substitutions, conservative substitutions are preferred in this invention. Particularly preferred, are amino acid substitutions where one, two or three amino acids are substituted by a conservative substitution. A “conservative substitution” is a substitution in which the substituting amino acid (naturally occurring or modified) has about the same steric and electronic properties as the amino acid being substituted. Thus, the substituting amino acid would have the same or a similar functional group in the side chain as the original amino acid.

A “conservative substitution” can also be achieved by utilizing a substituting amino acid that is identical to the amino acid being substituted except that a functional group in the side chain is fuctionalized with a suitable protecting group. Suitable protecting groups are described in Green and Wuts, “Protecting Groups in Organic Synthesis”, John Wiley and Sons, Chapters 5 and 7, 1991, the teachings of which are incorporated herein by reference. As with N-terminal and C-terminal protecting group, preferred protecting groups are those which facilitate transport of the peptide into a cell, for example, by reducing the hydrophilicity and increasing the lipophilicity of the peptide, and which can be cleaved in vivo, either by hydrolysis or enzymatically, inside the cell. (Ditter et al., J. Pharm. Sci. 57:783 (1968); Ditter et al., J. Pharm. Sci. 57:828 (1968); Ditter et al., J. Pharm. Sci. 58:557 (1969); King et al., Biochemistry 26:2294 (1987); Lindberg et al., Drug Metabolism and Disposition 17:311 (1989); and Tunek et al., Biochem. Pharm. 37:3867 (1988), Anderson et al., Arch. Biochem. Biophys. 239:538 (1985) and Singhal et al., FASEB J. 1:220 (1987)). Hydroxyl protecting groups include esters, carbonates and carbamate protecting groups. Amine protecting groups include alkoxy and aryloxy carbonyl groups, as described above for N-terminal protecting groups. Carboxylic acid protecting groups include aliphatic, benzylic and aryl esters, as described above for C-terminal protecting groups. In one embodiment, the carboxylic acid group in the side chain of one or more glutamic acid or aspartic acid residue in a peptide of the present invention is protected, preferably with a methyl, ethyl, benzyl or substituted benzyl ester, more preferably as a benzyl ester.

Provided below are groups of naturally occurring and modified amino acids in which each amino acid in a group has similar electronic and steric properties. Thus, a conservative substitution is made by substituting an amino acid with another amino acid from the same group. It is to be understood that these groups are non-limiting, i.e. that there are additional modified amino acids which could be included in each group.

Group I includes leucine, isoleucine, valine, methionine, phenylalanine, serine, cysteine, threonine and modified amino acids having the following side chains: ethyl, n-butyl, —CH₂CH₂OH, —CH₂CH₂CH₂OH,—CH₂CHOHCH₃ and —CH₂SCH₃. Preferably, Group I includes leucine, isoleucine, valine and methionine.

Group II includes glycine, alanine, valine, serine, cysteine, threonine and a modified amino acid having an ethyl side chain. Preferably, Group II includes glycine and alanine.

Group III includes phenylalanine, phenylglycine, tyrosine, tryptophan, cyclohexylmethyl, and modified amino residues having substituted benzyl or phenyl side chains. Preferred substituents include one or more of the following: halogen, methyl, ethyl, nitro, methoxy, ethoxy and —CN. Preferably, Group III includes phenylalanine, tyrosine and tryptophan.

Group IV includes glutamic acid, aspartic acid, a substituted or unsubstituted aliphatic, aromatic or benzylic ester of glutamic or aspartic acid (e.g., methyl, ethyl, n-propyl iso-propyl, cyclohexyl, benzyl or substituted benzyl), glutamine, asparagine, CO—NH-alkylated glutamine or asparagine (e.g., methyl, ethyl, n-propyl and iso-propyl) and modified amino acids having the side chain —(CH₂)₃—COOH, an ester thereof (substituted or unsubstituted aliphatic, aromatic or benzylic ester), an amide thereof and a substituted or unsubstituted N-alkylated amide thereof. Preferably, Group IV includes glutamic acid, aspartic acid, glutamine, asparagine, methyl aspartate, ethyl aspartate, benzyl aspartate and methyl glutamate, ethyl glutamate and benzyl glutamate.

Group V includes histidine, lysine, arginine, N-nitroarginine,β-cycloarginine, g-hydroxyarginine, N-amidinocitruline and 2-amino-4-guanidinobutanoic acid, homologs of lysine, homologs of arginine and omithine. Preferably, Group V includes histidine, lysine, arginine, and omithine. A homolog of an amino acid includes from 1 to about 3 additional methylene units in the side chain.

Group VI includes serine, threonine, cysteine and modified amino acids having C1-C5 straight or branched alkyl side chains substituted with —OH or —SH. Preferably, Group VI includes serine, cysteine or threonine.

In this invention any cysteine in the original sequence or subsequence can be replaced by a homocysteine or other sulfhydryl-containing amino acid residue or analog. Such analogs include lysine or beta amino alanine, to which a cysteine residue is attached through the secondary amino yielding lysine-epsilon amino cysteine or alanine-beta amino cysteine, respectively.

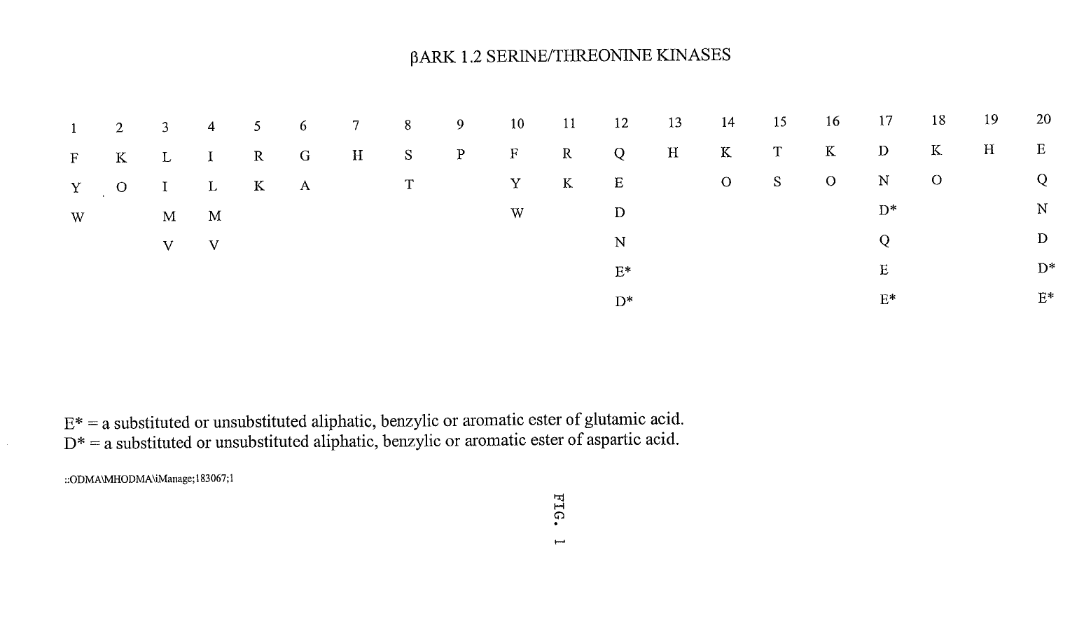

In another aspect, suitable substitutions for amino acid residues in the sequence of a HJ loop peptide include “severe substitutions” which result in peptide derivatives which modulate the activity of a GRK. Severe substitutions which result in peptide derivatives that inhibit the activity of a GRK are much more likely to be possible in positions which are not highly conserved throughout the family of peptides than at positions which are highly conserved. FIG. 1 shows the consensus sequences of the six to ten amino acids of the HJ loop peptides. Because D-amino acids have a hydrogen at a position identical to the glycine hydrogen side-chain, D-amino acids or their analogs can often be substituted for glycine residues.

A “severe substitution” is a substitution in which the substituting amino acid (naturally occurring or modified) has significantly different size, configuration and/or electronic properties compared with the amino acid being substituted. Thus, the side chain of the substituting amino acid can be significantly larger (or smaller) than the side chain of the amino acid being substituted and/or can have functional groups with significantly different electronic properties than the amino acid being substituted. Examples of severe substitutions of this type include the substitution of phenylalanine or cycohexylmethyl glycine for alanine, isoleucine for glycine, or —NH—CH[(—CH₂)₅—COOH]—CO— or aspartic acid. Alternatively, a functional group may be added to the side chain, deleted from the side chain or exchanged with another functional group. Examples of severe substitutions of this type include adding an amine or hydroxyl, carboxylic acid to the aliphatic side chain of valine, leucine or isoleucine, exchanging the carboxylic acid in the side chain of aspartic acid or glutamic acid with an amine or deleting the amine group in the side chain of lysine or ornithine. In yet another alternative, the side chain of the substituting amino acid can have significantly different steric and electronic properties from the functional group of the amino acid being substituted. Examples of such modifications include tryptophan for glycine, lysine for aspartic acid and —(CH₂)₄COOH for the side chain of serine. These examples are not meant to be limiting.

“Peptidomimetics” can be substituted for amino acid residues in the peptides of this invention. These peptidomimetics either replace amino acid residues or act as spacer groups within the peptides. The peptidomimetics often have steric, electronic or configurational properties similar to the replaced amino acid residues but such similarities are not necessarily required. The only restriction on the use of peptidomimetics is that the peptides retain their GRK modulating activity. Peptidomimetics are often used to inhibit degradation of the peptides by enzymatic or other degradative processes. The peptidomimetics can be produced by organic synthetic techniques. Examples of suitable peptidomimetics include D amino acids of the corresponding L amino acids, tetrazol (Zabrocki et al., J. Am. Chem. Soc. 110, 5875-5880 (1988)); isosteres of amide bonds (Jones et al., Tetrahedron Lett. 29, 3853-3856 (1988)); LL-3-amino-2-propenidone-6-carboxylic acid (LL-Acp) (Kemp et al., J. Org. Chem. 50, 5834-5838 (1985)). Similar analogs are shown in Kemp et al., Tetrahedron Lett. 29, 5081-5082 (1988) as well as Kemp et al., Tetrahedron Lett. 29, 5057-5060 (1988), Kemp et al., Tetrahedron Lett. 29, 4935-4938 (1988) and Kemp et al., J. Org. Chem.54, 109-115 (1987). Other suitable peptidomimetics are shown in Nagai and Sato, Tetrahedron Lett. 26, 647-650 (1985); Di Maio et al., J. Chem. Soc. Perkin Trans., 1687 (1985); Kahn et al., Tetrahedron Lett. 30, 2317 (1989); Olson et al., J. Am. Chem. Soc. 112, 323-333 (1990); Garvey et al., J. Org. Chem. 56, 436 (1990). Further suitable peptidomimetics include hydroxy-1,2,3,4-tetrahydroisoquinoline-3-carboxylate (Miyake et al., J. Takeda Res. Labs 43, 53-76 (1989)); 1,2,3,4-tetrahydroisoquinoline-3-carboxylate (Kazmierski et al., J. Am. Chem. Soc. 133, 2275-2283 (1991)); histidine isoquinolone carboxylic acid (HIC) (Zechel et al., Int. J. Pep. Protein Res. 43 (1991)); (2S, 3S)-methyl-phenylalanine, (2S, 3R)-methyl-phenylalanine, (2R, 3S)-methyl -phenylalanine and (2R, 3R)-methyl-phenylalanine (Kazmierski and Hruby, Tetrahedron Lett. (1991)).

The amino acid residues of the peptides can be modified by carboxymethylation, acylation, phosphorylation, glycosylation or fatty acylation. Ether bonds can be used to join the serine or threonine hydroxyl to the hydroxyl of a sugar. Amide bonds can be used to join the glutamate or aspartate carboxyl groups to an amino group on a sugar (Garg and Jeanloz, Advances in Carbohydrate Chemistry and Biochemistry, Vol. 43, Academic Press (1985); Kunz, Ang. Chem. Int. Ed. English 26, 294-308 (1987)). Acetal and ketal bonds can also be formed between amino acids and carbohydrates. Fatty acid acyl derivatives can be made, for example, by free amino group (e.g., lysine) acylation (Toth et al., Peptides: Chemistry, Structure and Biology, Rivier and Marshal, eds., ESCOM Publ., Leiden, 1078-1079 (1990)).

In one aspect, one, two or more of the amino acids in the sequence are modified with conservative substitutions; the substitutions can be in consensus positions, in non-consensus positions or in both. In another aspect, one, two or more of the amino acids in the sequence are modified with severe substitutions; the substitutions are preferably in non-consensus positions. FIG. 1 provides examples of conservative amino acid substitutions.

The peptides of this invention are most preferably modified by suitable protecting groups on the N-terminal amino acid such as acetyl, myristyl, stearyl, phenyl, adamantyl, naphthalyl, myristoleyl, toluyl, biphenyl, cinnamoyl, nitrobenzyl, benzoyl, furoyl, oleoyl, cyclohexyl, norboranyl, z-caproyl, ricinolelyl, and palmitoyl substituents. Particularly preferred are the addition of glycine, with one of those protecting groups substituted at the N-terminal amine of the glycine, to the N-terminus of the peptide. The C-terminal amino acid can be modified by suitable protecting groups such as by amidation. The interior amino acids of the peptides can have substituents added without loss of effectiveness and, in many instances, with enhanced effectiveness as well as, often, longer biological half-lives because they are not recognized or degraded by degradative enzymes or processes that exist in the individual or the prostate cancer cells. These substituents include benzylamide groups on lysine, biotinylation of lysine and diiodination of tyrosine. In addition, the D-isomer of any amino acid can be substituted for the natural L-isomer. This is particularly true for the lysine residues.

In this invention, particularly preferred peptides are those labeled as K024H001 (SEQ ID NO.:1), K024H003 (SEQ ID NO.:2), K024H007 (SEQ ID NO.:3), K024H101 (SEQ ID NO.:4), K024H102 (SEQ ID NO.:5), K024H103 (SEQ ID NO.:6), K024H104 (SEQ ID NO.:7), K024H105 (SEQ ID NO.:8), K024H106 (SEQ ID NO.:9), K024H107 (SEQ ID NO.:10), K024H108 (SEQ ID NO.:11), K024H109 (SEQ ID NO.:12), K024H110 (SEQ ID NO.:13), K024H111 (SEQ ID NO.:14), K024H112 (SEQ ID NO.:15), K024H113 (SEQ ID NO.:16), K024H114 (SEQ ID NO.:17), K024H901 (SEQ ID NO.:18), and K024H903 (SEQ ID NO.:19), as specified in FIG. 2.

The N-terminus and/or C-terminus of these peptides can be modified, as described above and as shown in FIG. 2. The N-terminal of these peptides is substituted and the C-terminal is amidated. Other protecting groups for amides and carboxylic acids can be used, as described above. Optionally, one or both protecting groups can be omitted. The peptides may be linear or cyclic.

Also included are peptides having the sequence of: K024H001 (SEQ ID NO.:1), K024H003 (SEQ ID NO.:2), K024H007 (SEQ ID NO.:3), K024H101 (SEQ ID NO.:4), K024H102 (SEQ ID NO.:5), K024H103 (SEQ ID NO.:6), K024H104 (SEQ ID NO.:7), K024H105 (SEQ ID NO.:8), K024H106 (SEQ ID NO.:9), K024H107 (SEQ ID NO.:10), K024H108 (SEQ ID NO.:11), K024H109 (SEQ ID NO.:12), K024H110 (SEQ ID NO.:13), K024H111 (SEQ ID NO.:14), K024H112 (SEQ ID NO.:15), K024H113 (SEQ ID NO.:16), K024H114 (SEQ ID NO.:17), K024H901 (SEQ ID NO.:18), and K024H903 (SEQ ID NO.:19) as specified in FIG. 2, with the proviso that any one or two of the amino residues in the peptide can vary, being replaced by any naturally occurring amino acid or analog thereof.

The present invention also includes cyclic peptides having amino acid sequences corresponding to a modified sequence of the HJ loop peptides. These cyclic peptides inhibit the activity of GRKs.

A “cyclic peptide” refers, in one instance, to a peptide or peptide derivative in which a ring is formed by the formation of a peptide bond between the nitrogen atom at the N-terminus and the carbonyl carbon at the C-terminus.

“Cyclized” also refers to the forming of a ring by a covalent bond between the nitrogen at the N-terminus of the compound and the side chain of a suitable amino acid in the peptide, preferably the side chain of the C-terminal amino acid. For example, an amide can be formed between the nitrogen atom at the N-terminus and the carbonyl carbon in the side chain of an aspartic acid or a glutamic acid. Alternatively, the peptide or peptide derivative can be cyclized by forming a covalent bond between the carbonyl at the C-terminus of the compound and the side chain of a suitable amino acid in the peptide, preferably the side chain of the N-terminal amino acid. For example, an amide can be formed between the carbonyl carbon at the C-terminus and the amino nitrogen atom in the side chain of a lysine or an ornithine. Additionally, the peptide or peptide derivative can be cyclized by forming an ester between the carbonyl carbon at the C-terminus and the hydroxyl oxygen atom in the side chain of a serine or a threonine. “Cyclized” also refers to forming a ring by a covalent bond between the side chains of two suitable amino acids in the peptide, preferably the side chains of the two terminal amino acids. For example, a disulfide can be formed between the sulfur atoms in the side chains of two cysteines. Alternatively, an ester can be formed between the carbonyl carbon in the side chain of, for example, a glutamic acid or an aspartic acid, and the oxygen atom in the side chain of, for example, a serine or a threonine. An amide can be formed between the carbonyl carbon in the side chain of, for example, a glutamic acid or an aspartic acid, and the amino nitrogen in the side chain of, for example, a lysine or an ornithine.

In addition, a peptide or peptide derivative can be cyclized with a linking group between the two termini, between one terminus and the side chain of an amino acid in the peptide or peptide derivative, or between the side chains to two amino acids in the peptide or peptide derivative. Suitable linking groups are disclosed in Lobl et al., WO 92/00995 and Chiang et al., WO 94/15958, the teachings of which are incorporated into this application by reference.

There are many suitable substances which can be employed in the methods of the invention, particularly as inhibitors of GRK2 or GRK3. For example, low molecular weight organic molecules can act as inhibitors of GRK2 or GRK3. Such low molecular weight organic molecules are known in the art. Preferred low molecular weight organic molecules for use with the present invention are those that specifically inhibit the activity of GRK2 or GRK3.

Another type of inhibitor of GRK2 or GRK3 is anti-sense nucleic acids. The nucleic acids are single stranded ribonucleic or deoxyribonucleic acid strands which contain several (tens) nucleotides joined together through normal sugar-phosphate bonds. These strands bind via hybridization within or upstream of the structural gene, that encodes GRK2 or GRK3. This hybridization binding blocks proper transcription or expression of GRK2 or GRK3 from occurring in individuals suffering from Syndrome X or Type II diabetes. Since proper transcription or expression is effectively blocked by the hybridization of the anti-sense nucleic acids to the DNA or RNA that contains the GRK2 or GRK3 structural gene, inhibition of the kinase is effected. There is much less active GRK2 or GRK3 produced in the presence than without the presence of these antisense nucleic acids. This diminution of the amount of active GRK2 or GRK3 necessarily is manifested as an inhibition of GRK2 or GRK3 and results in increased activity of the interacting 7TM receptor. The particular nucleotides that are joined together to form the anti-sense nucleic acids are those that are complementary to the region of the GRK2 or GRK3 structural gene. Thus, the anti-sense nucleic acids are complementary to the region of the GRK2 or GRK3 to which the anti-sense nucleic acids bind via hybridization. These nucleotides of the anti-sense nucleic acids are specifically determined by the nucleotides of the target location and can easily be identified by the skilled practitioner once the sequence of the target location is established. The target location is a matter of choice to some extent. It lies within the region of the structural gene that encodes GRK2 or GRK3 or is upstream of this coding region in the recognition or regulation region of the GRK2 or GRK3 gene. The target location nucleotide sequence can easily be established by the skilled practitioner from publicly available information concerning the GRK2 or GRK3 gene or can be obtained by routine examination of homologous genes coupled with standard molecular biology techniques.

Still another type of inhibitor of GRK2 or GRK3 is negative dominant GRK2 or GRK3 genes. The presence of these genes in individuals suffering from Syndrome X or Type II diabetes allows non-functional GRK2 or GRK3 to be expressed to the exclusion of functional GRK2 or GRK3. The negative dominant GRK2 or GRK3 in these individuals is inhibitory of GRK2 or GRK3 activity because this kinase is non-functional. Non-functional kinases, by definition, have no kinase activity. Negative dominant GRK2 or GRK3 genes can be administered into the individual's cells by gene transfer techniques, which are becoming increasingly more standard in the art (calcium precipitation, electrical discharge, physical injection, use of carriers such as recombinant vectors, etc.). The introduced negative dominant GRK2 or GRK3 gene is incorporated in the cell genome. There, copies of it are passed to progeny cells. Since the GRK2 or GRK3 gene is negative dominant, it will be expressed in response to signals which induce GRK2 or GRK3 expression rather than the active form of GRK2 or GRK3. Cells which have incorporated the negative dominant GRK2 or GRK3 gene will have little or no GRK activity because the expressed GRK2 or GRK3 is inactive. The negative dominant GRK2 or GRK3 genes can be found in the art or can be produced by standard gene mutation techniques which are well known to skilled practitioners in the art. These genes can be suitably packaged for transgenic procedures by appropriate methods and materials known to the skilled practitioners.

A further type of inhibition of GRK2 or GRK3 is antibodies that are immunoreactive with GRK2 or GRK3. These antibodies bind to the kinase and thereby severely limit or prohibit its kinase activity. The antibodies can be of any class or type. The binding side of the antibodies can be anywhere on the GRK2 or GRK3 molecule provided the immunoreactive binding between the antibody and the kinase molecule results in a severe inhibition of GRK2 or GRK3 activity. The antibodies can be polyclonal or monoclonal and are produced by well-known techniques to the skilled practitioner by using the GRK2 or GRK3 or an immunogenic fragment thereof as the antigenic stimulus. The antibodies can be delivered to the individual's cells by depositing suitable clonal cells, which produce the antibodies, into the individual who is suffering from syndrome X or from Type II diabetes or who is susceptible to developing these medical indications. These clonal cells secrete the antibodies into the bloodstream where they are carried to other cells for immunoreaction with the GRK2 or GRK3 molecules. Binding fragments of antibodies are also suitable provided they bind GRK2 or GRK3 with sufficient affinity that the activity of the kinase is at least severely limited. Alternatively, the antibodies or suitable binding fragments can be introduced into the individual's cells or administered to the individual by any of a variety of techniques known to the skilled practitioner (physical injection, attachment to carriers that cross cell membranes, transgenic introduction into the cells for subsequent induction of expression, etc.). The secreted, introduced or expressed antibodies or suitable antibody fragments thereof immunoreactively bind to the GRK2 or GRK3 molecules, thereby inhibiting their activity.

A further type of inhibitor of GRK2 or GRK3 is peptides. Preferred are the peptides described above, which herein are designated as GRK2-or GRK3-derived HJ loop peptides. These peptides are the preferred embodiment of this invention as inhibitors of GRK2 or GRK3 and thereby suitable in treating syndrome X or Type II diabetes mellitus. The peptides apparently bind to GRK2 or GRK3 and inhibit the activity of this kinase. This GRK2 or GRK3 kinase inhibition causes an increase in the responsiveness of the interacting 7TM receptor. Quite often Syndrome X or type II diabetes is (are) eliminated. The peptides can be produced by a variety of techniques known to the skilled practitioner including organic synthetic procedures and production from cells that contain one or more genes that encode these peptides. Thee genes can be incorporated in the host cells by recombinant techniques.

The activity of a GRK is “modulated” or “altered” when the activity of the GRK is increased or decreased. An increase or decrease in the activity of a GRK can be detected by assessing in vitro the extent of phosphorylation of a protein substrate of the GRK being tested or by a corresponding modulation, increase or decrease, in a cellular activity or function which is under the control of the GRK. Examples of these cellular functions include cell proliferation, cell differentiation, cell morphology, cell survival or apoptosis, cell response to external stimuli, gene expression, lipid metabolism, glycogen metabolism and mitosis.

It can be readily determined whether a substance, such as, for example, the peptides described above, modulates (in particular, inhibits) the activity of a GRK by incubating the substance with cells which have one or more cellular activities controlled by a 7TM receptor that interacts with a GRK. The cells are incubated with the substance to produce a test mixture under conditions suitable for assessing the activity of the GRK. The activity of the GRK is assessed and compared with a suitable control, e.g., the activity of the same cells incubated under the same conditions in the absence of the substance. A lesser activity of the GRK in the test mixture compared with the control indicates that the test substance, for instance a test peptide inhibits the activity of the GRK. Alternatively, an increased activity of the GRK in the test mixture compared with the control indicates that the test substance, for instance a test peptide, enhances the activity of the GRK. No change in activity of the GRK in the test mixture compared with the control indicates absence of modulation by the test substance.

Conditions suitable for assessing GRK activity include conditions suitable for assessing a cellular activity or function under control of the 7TM receptor that interacts with a GRK. Generally, a cellular activity or function can be assessed when the cells are exposed to conditions suitable for cell growth, including a suitable temperature (for example, between about 30° C. to about 42° C.) and the presence of the suitable concentrations of nutrients in the medium (e.g., amino acids, vitamins, growth factors).

Generally, the activity of the GRK in the test mixture is assessed by making a quantitative measure of the cellular activity which the GRK controls. The cellular activity can be, for example, melanin production. GRK activity is assessed by measuring melanin production, for example, by comparing the amount of melanin present after a given period of time with the amount of melanin originally present.

In a preferred embodiment of the invention, modulation of GRK activity is determined by measuring melanogenesis by melanocytes in cell cultures, as further described below. It is to be understood that the assay described herein for determining whether a substance such as one of the peptides described above modulates a cellular activity or function under the control of a GRK can be performed with cells other than those specifically described herein.

The present invention is directed to methods of modulating the activity of a 7TM receptor that interacts with a GRK in a subject. A “subject” is preferably a human, but can also be animals in need of treatment, e.g., veterinary animals (e.g., dogs, cats, and the like), farm animals (e.g., cows, pigs, horses, chickens and the like) and laboratory animals (e.g., rats, mice, guinea pigs and the like).

A “therapeutically effective amount” is the quantity of compound which results in an improved clinical outcome as a result of the treatment compared with a typical clinical outcome in the absence of the treatment. An “improved clinical outcome” results in the individual with the disease experiencing fewer symptoms or complications of the disease, including a longer life expectancy, as a result of the treatment. With respect to diabetes, an improved clinical outcome refers to a longer life expectancy, a reduction in the complications of the disease (e.g., neuropathy, retinopathy, nephropathy and degeneration of blood vessels) and an improved quality of life, as described above. Another aspect of an improved clinical outcome is a reduction in medication dosage (e.g., a reduction in insulin or other hypoglycemic agent needed to maintain adequate blood glucose levels).

With respect to obesity, an improved clinical outcome refers to increased weight reduction per calory intake. It also refers to a decrease in the complications which are a consequence of obesity, for example heart disease such as arteriosclerosis and high blood pressure. With respect to syndrome X an improved clinical outcome refers to a longer life expectancy, a reduction in the incidence or severity of the different mobidities included in the syndrome (e.g., ischemic heart disease, atherosclerosis, type II DM and obesity) and an improved quality of life.

The amount of substance, e.g. a peptide such as a GRK-derived HJ loop peptide, described above, administered to the individual will depend on the type and severity of the disease and on the characteristics of the individual, such as general health, age, sex, body weight and tolerance to drugs. The skilled artisan will be able to determine appropriate dosages depending on these and other factors. Typically, a therapeutically effective amount of the peptide or peptide derivative can range from about 1 mg per day to about 1000 mg per day for an adult. Preferably, the dosage ranges from about 1 mg per day to about 100 mg per day.

The peptide and peptide derivatives of the present invention are preferably administered parenterally. Parenteral administration can include, for example, systemic administration, such as by intramuscular, intravenous, subcutaneous, or intraperitoneal injection. Peptides or peptide derivatives which resist proteolysis can be administered orally, for example, in capsules, suspensions or tablets. The peptide or peptide derivative can also be administered by inhalation or insufflation or via a nasal spray.

The substance can be administered to the individual in conjunction with an acceptable pharmaceutical carrier as part of a pharmaceutical composition for treating the diseases discussed above. Suitable pharmaceutical carriers may contain inert ingredients which do not interact with the peptide or peptide derivative. Standard pharmaceutical formulation techniques may be employed such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., Controlled Release of Biological Active Agents, John Wiley and Sons, 1986).

Of particular interest herein also relates to effecting an enhanced signal by a 7TM receptor by the local inhibition of a GRK. For example, MSH triggers melanin formation by melanocytes. It is therefore predicted with this invention that GRK inhibitors will enhance melanogenesis by low-levels of MSH. Accordingly, in one embodiment of the invention, the method includes local administration of the substance to an individual in need of enhanced or reduced signaling of a 7TM receptor. For instance, local administration of a substance which inhibits GRK to an individual, down regulates MSH, resulting in an increase in melanogenesis.

The peptide and peptide derivatives of the present invention have many utilities other than as a therapeutic agent. Some of these uses are discussed in the following paragraphs.

The GRK-derived HJ loop peptides of the present invention can be useful in the preparation of specific antibodies against GRKs. Suitable antibodies can be raised against a HJ loop peptide by conjugating the peptide to a suitable carrier, such as keyhole limpet hemocyanin or serum albumin; polyclonal and monoclonal antibody production can be performed using any suitable technique. A variety of methods have been described (see e.g., Kohler et al., Nature, 256: 495-497 (1975) and Eur. J. Immunol. 6: 511-519 (1976); Milstein et al., Nature 266: 550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer 1994), Ausubel, F. M. et al., Eds., (John Wiley & Sons: New York, N.Y.), Chapter 11, (1991)). Generally, a hybridoma can be produced by fusing a suitable immortal cell line (e.g., a myeloma cell line such as SP2/0) with antibody producing cells. The antibody producing cell, preferably those of the spleen or lymph nodes, can be obtained from animals immunized with the antigen of interest.

The fused cells (hybridomas) can be isolated using selective culture conditions, and cloned by limiting dilution. Cells which produce antibodies with the desired specificity can be selected by a suitable assay (e.g., ELISA).

Antibodies, including monoclonal antibodies, against HJ loop peptides have a variety of uses. For example, those against or reactive with GRKs can be used to identify and/or sort cells exhibiting that kinase on the cell surface (e.g., by means of fluorescence activated cell sorting or histological analyses). Monoclonal antibodies specific for a GRK can also be used to detect and/or quantitate the kinase expressed on the surface of a cell or present in a sample (e.g., in an ELISA). Alternatively, the antibodies can be used to determine if an intracellular a GRK is present in the cytoplasm of the cell. A lysate of the cell is generated (for example, by treating the cells with sodium hydroxide (0.2 N) and sodium dodecyl sulfate (1%) or with a non-ionic detergent like NP-40, centrifugating and separating the supernatant from the pellet), and treated with anti-HJ loop peptide antibody specific for a GRK. The lysate is then analyzed, for example, by Western blotting or immunoprecipitation for complexes between a GRK and antibody. Anti-HJ loop peptide antibodies can be utilized for the study of the intracellular distribution (compartmentalization) of GRKs under various physiological conditions via the application of conventional immunocytochemistry such as immunofluorescence, immunoperoxidase technique and immunoelectron microscopy, in conjunction with the specific anti-HJ loop peptide antibody. Antibodies reactive with the HJ loop peptides are also useful to detect and/or quantitate the GRKs in a sample, or to purify the GRKs (e.g., by immunoaffinity purification).

The GRK-derived HJ loop peptides of the present invention can also be used to identify ligands which interact with GRKs and which inhibit the activity of GRKs. For example, an affinity column can be prepared to which a HJ loop peptide is covalently attached, directly or via a linker. This column, in turn, can be utilized for the isolation and identification of specific ligands which bind the HJ loop peptide and which will also likely bind the GRK. The ligand can then be eluted from the column, characterized and tested for its ability to modulate GRK function.

Peptide sequences in the compounds of the present invention may be synthesized by solid phase peptide synthesis (e.g., t-BOC or F-MOC) method, by solution phase synthesis, or by other suitable techniques including combinations of the foregoing methods. The t-BOC and F-MOC methods, which are established and widely used, are described in Merrifield, J. Am. Chem. Soc. 88:2149 (1963); Meienhofer, Hormonal Proteins and Peptides, C. H. Li, Ed., Academic Press, 1983, pp. 48-267; and Barany and Merrifield, in The Peptides, E. Gross and J. Meienhofer, Eds., Academic Press, New York, 1980, pp. 3-285. Methods of solid phase peptide synthesis are described in Merrifield, R. B., Science, 232: 341 (1986); Carpino, L. A. and Han, G. Y., J. Org. Chem., 37: 3404 (1972); and Gauspohl, H. et al., Synthesis, 5: 315 (1992)). The teachings of these references are incorporated herein by reference.

Methods of cyclizing compounds having peptide sequences are described, for example, in Lobl et al., WO 92/00995, the teachings of which are incorporated herein by reference. Cyclized compounds can be prepared by protecting the side chains of the two amino acids to be used in the ring closure with groups that can be selectively removed while all other side-chain protecting groups remain intact. Selective deprotection is best achieved by using orthogonal side-chain protecting groups such as allyl (OAI) (for the carboxyl group in the side chain of glutamic acid or aspartic acid, for example), allyloxy carbonyl (Aloc) (for the amino nitrogen in the side chain of lysine or omithine, for example) or acetamidomethyl (Acm) (for the sulfhydryl of cysteine) protecting groups. OAI and Aloc are easily removed by Pd^{o} and Acm is easily removed by iodine treatment.

The invention is illustrated by the following examples which are not intended to be limiting in any way.

### EXAMPLE 1

Preparation of GRK-derived HJ Loop Peptides

The compounds of this invention can be synthesized utilizing a 430A Peptide Synthesizer from Applied Biosystems using F-Moc technology according to manufacturer's protocols. Other suitable methodologies for preparing peptides are known to person skilled in the art. See e.g., Merrifield, R. B., Science, 232: 341 (1986); Carpino, L. A., Han, G. Y., J. Org. Chem., 37: 3404 (1972); Gauspohl, H., et al., Synthesis, 5: 315 (1992)). The teachings of which are incorporated herein by reference. Rink Amide Resin [4(2′,4′ Dimethoxyphenyl-FMOC amino methyl) phenoxy resin] was used for the synthesis of C-amidated peptides. The alpha-amino group of the amino acid was protected by an FMOC group, which was removed at the beginning of each cycle by a weak base, 20% piperidine in N-methylpyrrolidone (NMP). After deprotection, the resin was washed with NMP to remove the piperidine. In situ activation of the amino acid derivative was performed by the FASTMOC Chemistry using HBTU (2(1-benzotriazolyl-1-yl)-1,1,3,3-tetramethyluronium) dissolved in HOBt (1-hydroxybenzotriazole) and DMF (dimethylformamide). The amino acid was dissolved in this solution with additional NMP. DIEA (diisopropylethylamine) was added to initiate activation. Alternatively, the activation method of DCC (dicycbohexylcarbodiimide) and HOBL was utilized to form an HOBt active ester. Coupling was performed in NMP. Following acetylation of the N-terminus (optional), TFA (trifluoroacetic acid) cleavage procedure of the peptide from the resin and the side chain protecting groups was applied using 0.75 g crystalline phenol; 0.25 ml EDT (1,2-ethandithiol); 0.5 ml thioanisoie; 0.5 ml D.I. H₂O; 10 ml TFA.

### EXAMPLE 2

### Type II Diabetes in Sand Rats (psamomys)

Sand-rats (psamomys) which are genetically prone to develop Type II diabetes were used in this study. The genetically selected sand-rats, 3 to 6 months old, were fed an energy-rich diet (Weizmann H E) for about 3 to 10 days until they became diabetic, as judged by their elevated blood-glucose level (see R. Kalman et al., “The Efficiency of Sand Rat Metabolism is Responsible for Development of Obesity and Diabetes”, J. of Basic & Clinical Physiology & Pharmacology (1993), vol. 4, no. 1-2, pp 57-68, the pertinent portions of which are incorporated herein by reference).

The diabetic sand-rats were injected i.p. once a week with GRK-derived peptide K024H107 at a dose of 10 mg/kg. The peptide was prepared by diluting a 10 mM solution of the peptide in 100% DMSO with phosphate buffered saline (PBS) containing 0.1% bovine serum albumin (BSA) to a concentration of 400 μM. Forty μM of the 10 mM peptide in DMSO solution was mixed with 160 μl of 1.6M NH₄HCO₃ and heated for 40 minutes at 100° C. The resultant solution was then diluted to 400 μM in 2 M Hepes buffer (pH 7.0). This peptide stock solution was labeled “tbi”. The vehicle of the solution for injection included 8% DMSO, 0.67M ammonium bicarbonate, and 2M Hepes. Control animals received an i.p. injection of the vehicle only.

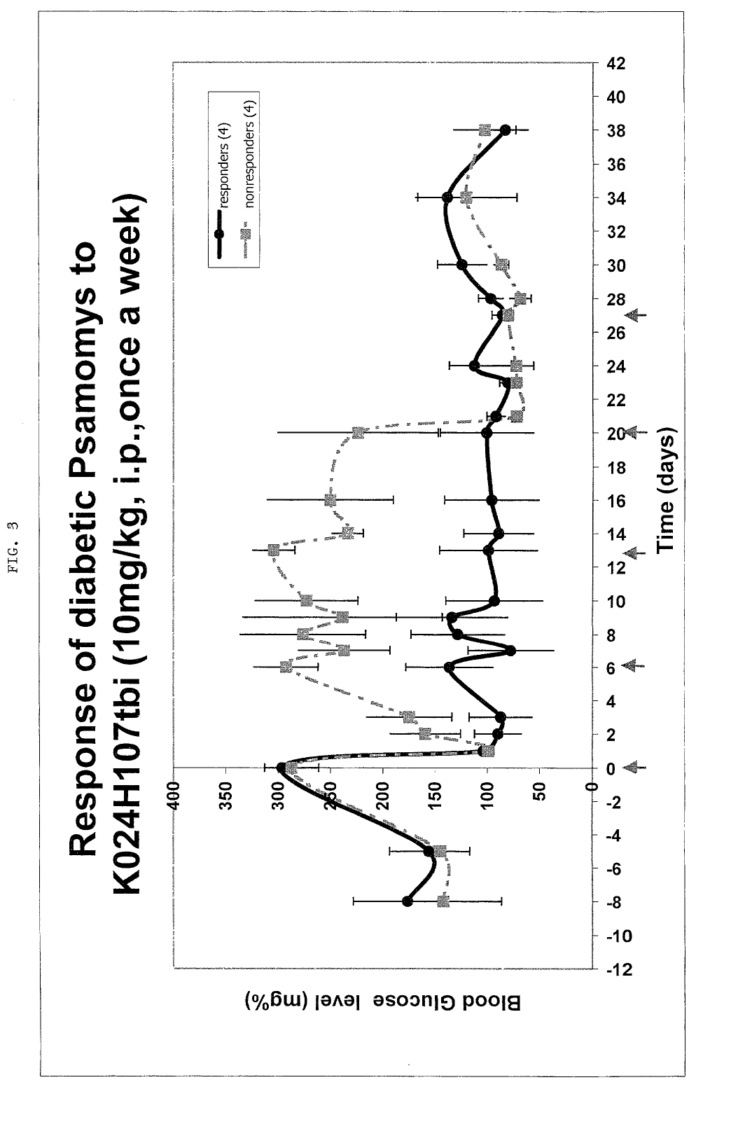

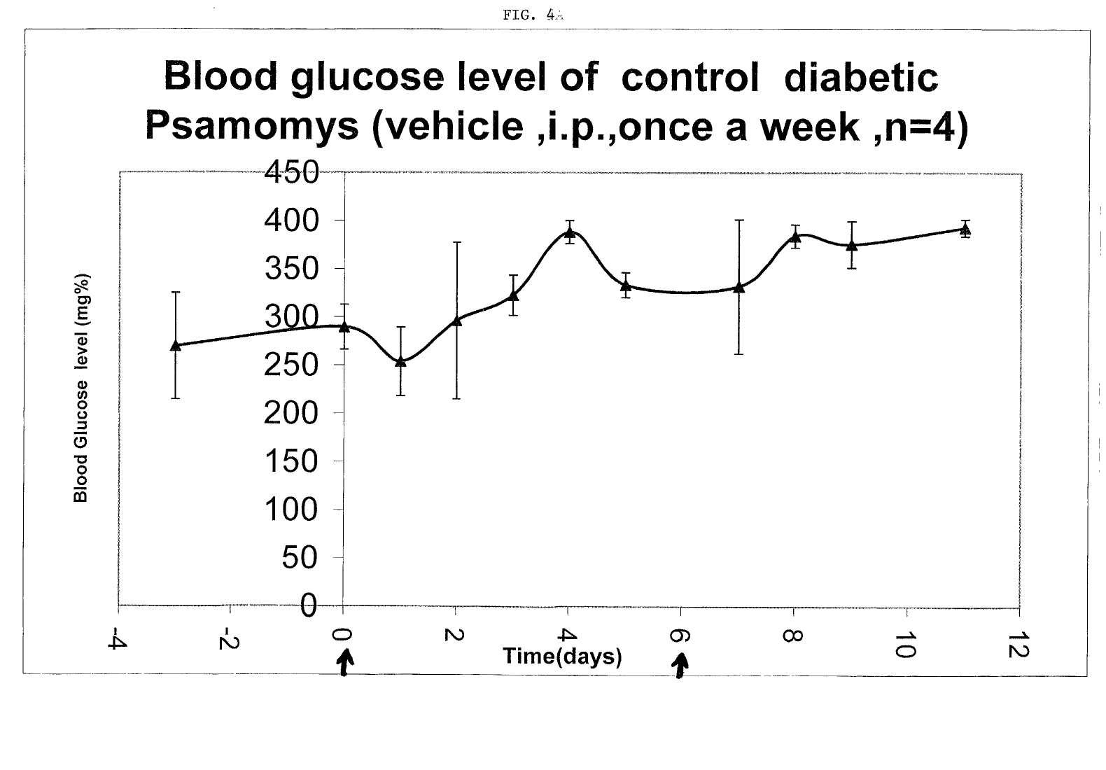

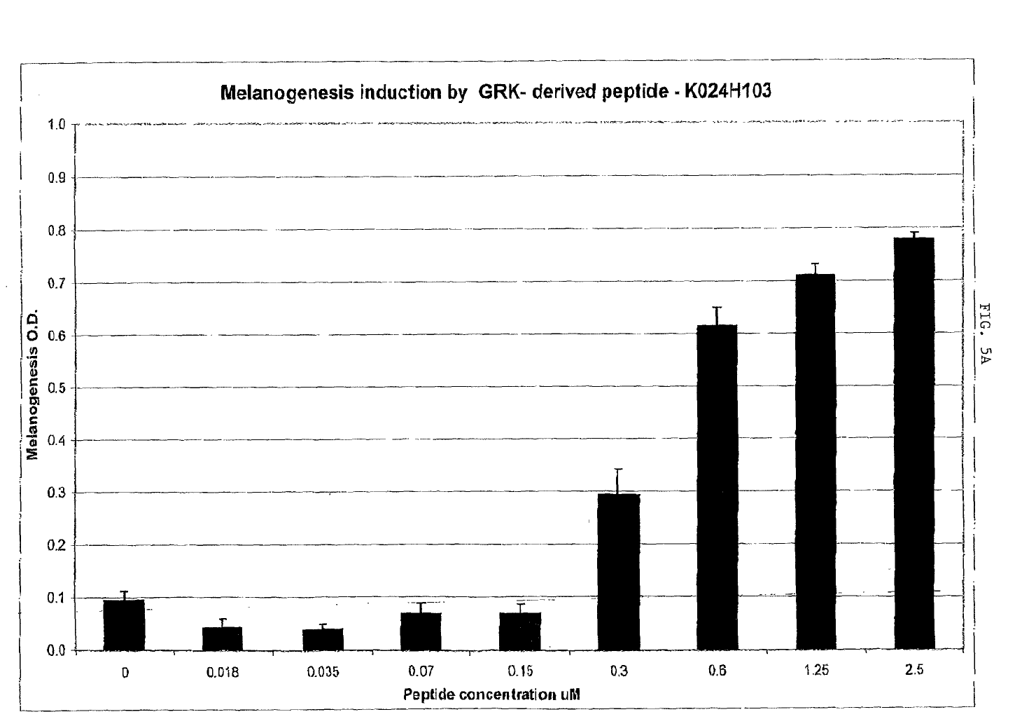

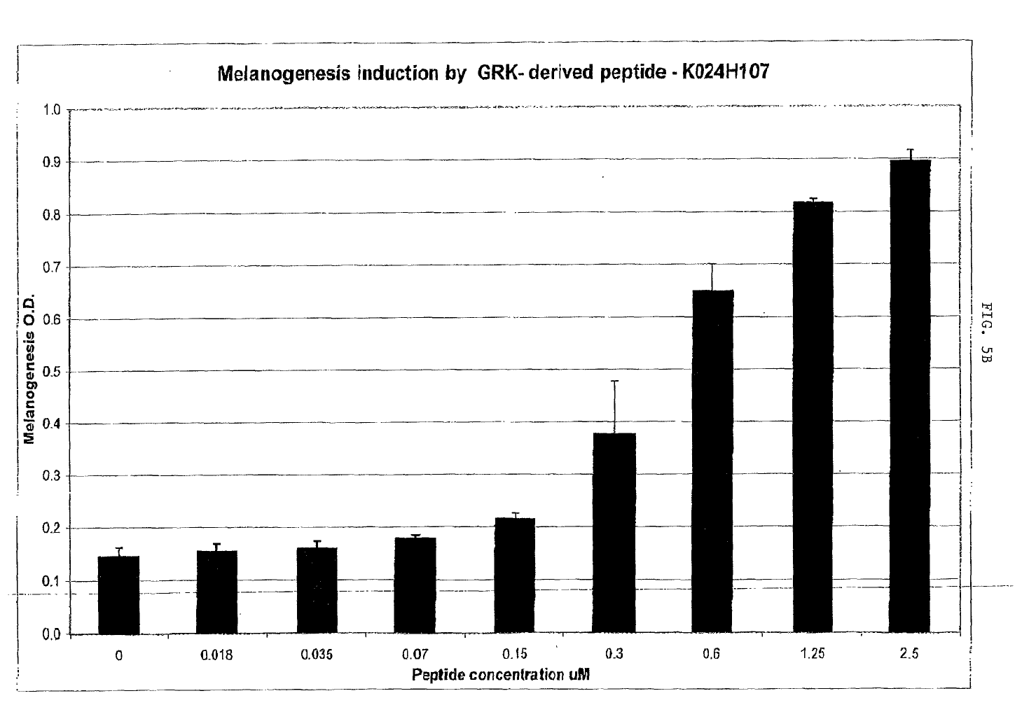

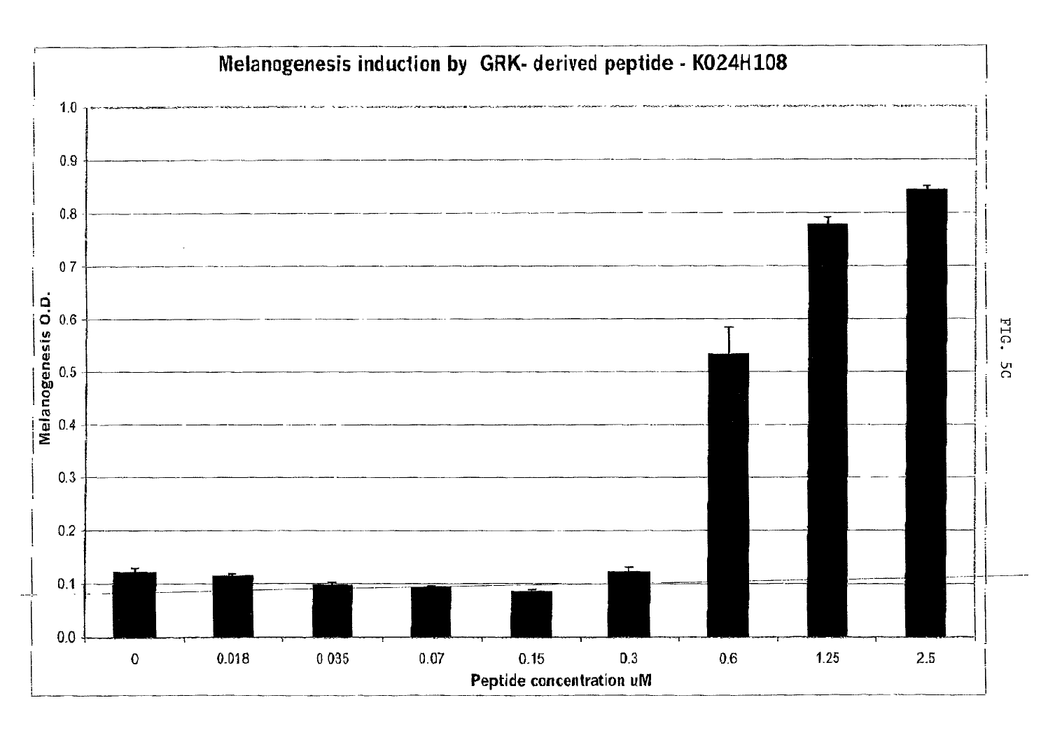

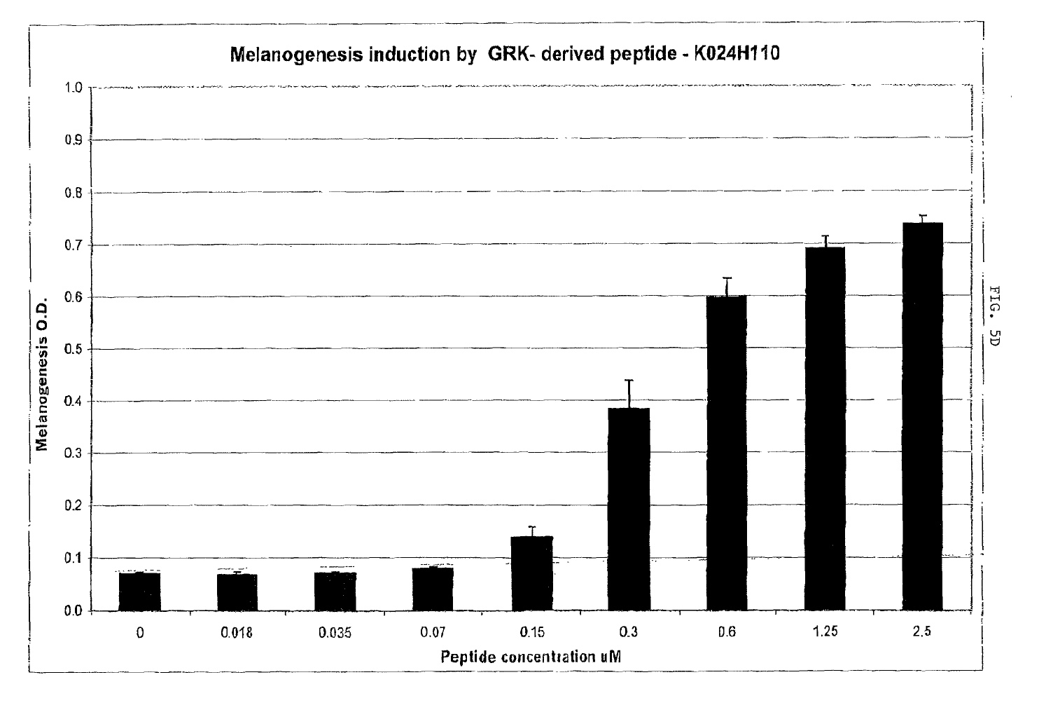

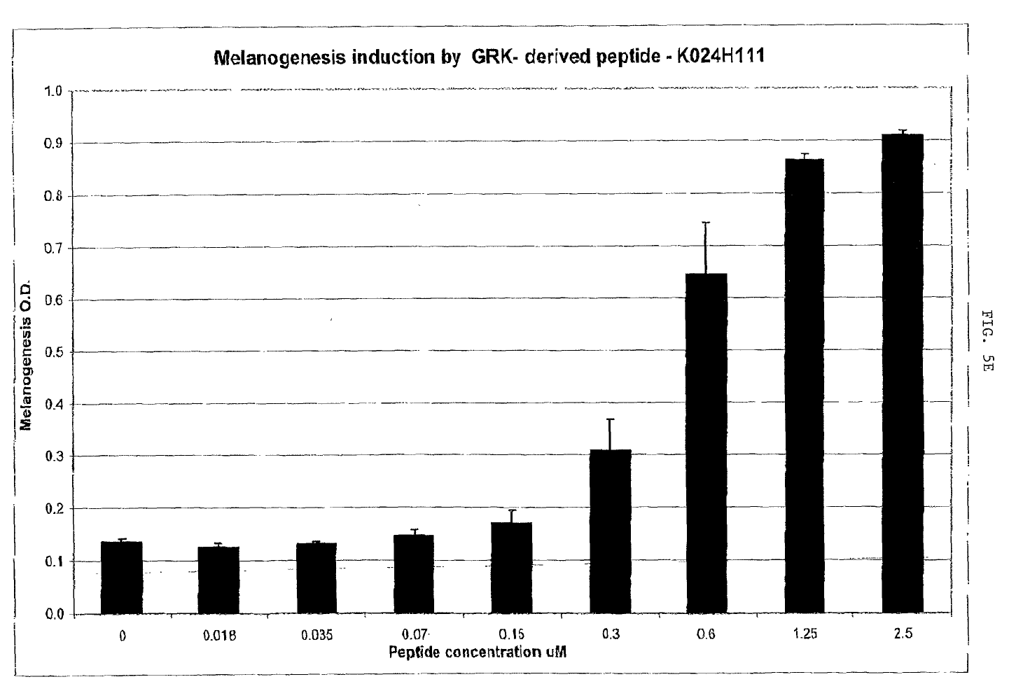

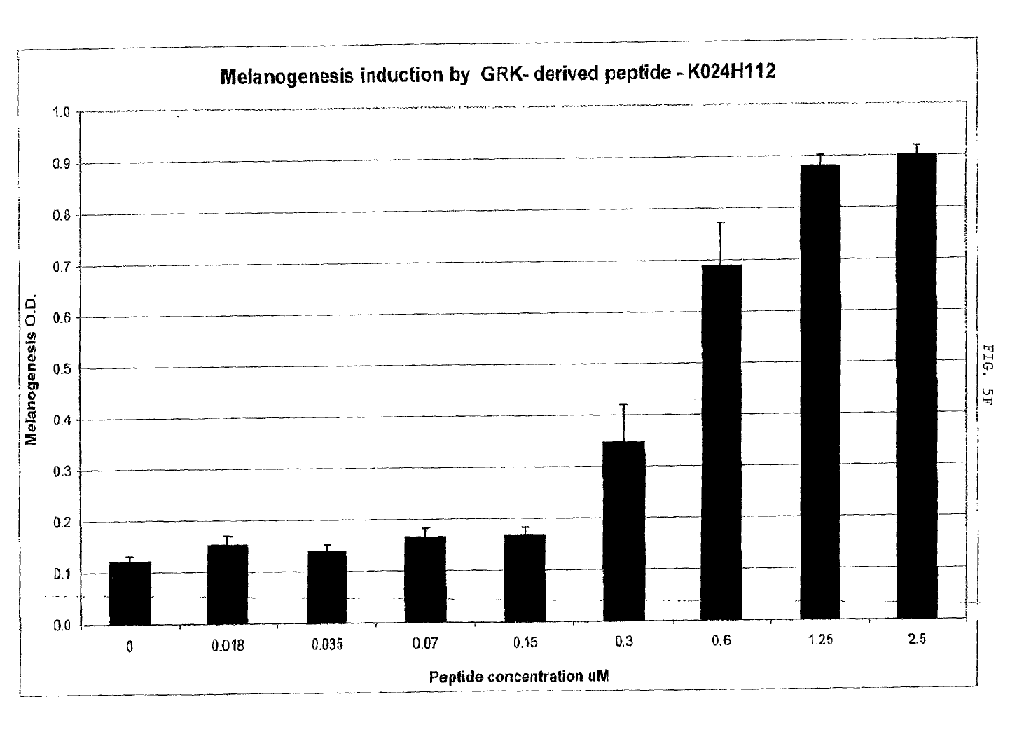

As can be seen from FIGS. 3 and 4, after a single injection of the GRK-derived peptide there was dramatic decrease in blood-glucose to the normal level (FIG. 3), while no change was observed in the controls (FIG. 4). Additionally, in the treated group, it was noted that 4 animals became normoglycemic already after the first injection (responders, FIG. 3), and the rest of the treated animals also became normoglycemic after three additional weekly injections (“nonresponders”, FIG. 3).

### EXAMPLE 3

### Measurement of Melanogenesis by Melanocytes in Cell Culture

Murine B16 melanoma cells were grown in DMEM+10% FCS+2 mM Glutamine+100 units/ml Penicillin+0.1 mg/ml Streptomycin. The cells were incubated under controlled conditions (37° C., 5% CO₂).

The melanoma cells were plated in 96-well microtiter plates, 5,400 cells per well, and allowed to grow for 24 hours. Selected GRK-derived peptides were solubilized in DMSO and then diluted in PBS+0.1% BSA to 10×of the final concentration (see the procedure in Example 2).

The peptides were added to the corresponding wells at the stated final concentrations (see FIGS. 4A-4F). The vehicle containing equal concentrations of DMSO, PBS and BSA was used as the control. The cells were then incubated for an additional 4-days, when dark melanin pigment accumulated in the wells of the treated cells.

Melanogenesis was then assessed by addition of 70 μl IN NaOH per well to release all melanin from the cells and the optical density was determined by 405 nm, using an ELX-800 ELISA plate reader. Six wells were used for each concentration. The results are shown in FIGS. 4A-4F. It can be seen from these graphs that significant melanogenesis occurred at peptide concentrations of 0.6 μM and, for some peptides, as low as 0.15 μM. It is readily apparent from these graphs that these peptides cause an enhancement in melanogenesis from melanocytes.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of modulating metabolism in an individual comprising the administration of a substance which alters G-protein-coupled receptor kinase 2 (GRK2) activity or G-protein-coupled receptor kinase 3 (GRK3) activity in said individual, wherein said administration results in an increase or a decrease of said metabolism.

2. The method of claim 1 wherein said GRK interacts with a seven transmembrane receptor(7TM), thereby affecting the ability of said receptor to carry out its function when its complementary ligand is present and said activity of said GRK is altered.

3. The method of claim 2 wherein said activity of said GRK is inhibited.

4. The method of claim 3 wherein said inhibition of said GRK enhances said ability of said receptor to carry out its function when its complementary ligand is present.

5. The method of claim 4 wherein said substance that inhibits the activity of said GRK is a peptide.

6. The method of claim 5 wherein the modulated metabolism is selected from the group consisting of enhanced melanogenesis, alleviation of syndrome X, corrected diabetes mellitus Type II, improvement of heart function, relief of hypertension and lower propensity for obesity.

7. The method of claim 5 wherein said peptide is selected from the group consisting K024H001 (SEQ ID NO.:1), K024H003 (SEQ ID NO.:2), K024H007 (SEQ ID NO.:3), K024H101 (SEQ ID NO.:4), K024H102 (SEQ ID NO.:5), K024H103 (SEQ ID NO.:6), K024H104 (SEQ ID NO.:7), K024H105 (SEQ ID NO.:8), K024H106 (SEQ ID NO.:9), K024H107 (SEQ ID NO.:10), K024H108 (SEQ ID NO.:11), K024H109 (SEQ ID NO.:12), K024H110 (SEQ ID NO.:13), K024H111 (SEQ ID NO.:14), K024H112 (SEQ ID NO.:15), K024H113 (SEQ ID NO.:16), K024H1114 (SEQ ID NO.:17), K024H901 (SEQ ID NO.:18), and K024H903 (SEQ ID NO.:19).

8. A method of modulating activity of a G-protein coupled receptor kinase (GRK) in a laboratory animal or in an individual suffering from type II diabetes, obesity or syndrome X comprising administering to said laboratory animal or said individual a GRK-derived HJ loop peptide.

9. The method of claim 8 wherein the GRK is selected from a bARK1 or a bARK2 kinase family.

10. The method of claim 9 wherein the GRK is a bARK1 kinase.

11. The method of claim 8 wherein the GRK-derived HJ loop peptide is selected form the group consisting K024H001 (SEQ ID NO.:1), K024H003 (SEQ ID NO.:2), K024H007 (SEQ ID NO.:3), K024H101 (SEQ ID NO.:4), K024H102 (SEQ ID NO.:5), K024H103 (SEQ ID NO.:6), K024H104 (SEQ ID NO.:7), K024H105 (SEQ ID NO.:8), K024H106 (SEQ ID NO.:9), K024H107 (SEQ ID NO.:10), K024H108 (SEQ ID NO.:11), K024H109 (SEQ ID NO.:12), K024H110 (SEQ ID NO.:13), K024H111 (SEQ ID NO.:14), K024H112 (SEQ ID NO.:15), K024H113 (SEQ ID NO.:16), K024H114 (SEQ ID NO.:17), K024H901 (SEQ ID NO.:18), and K024H903 (SEQ ID NO.:19).

12. The method of claim 8 wherein the individual suffers from syndrome X.

13. The method of claim 8 wherein the individual suffers from Type II diabetes mellitus.

14. The method of claim 8 wherein the laboratory animal is a sand rat (Psamomys obesus).

15. The method of claim 8 wherein said administering the GRK-derived HJ loop peptide is systemic.

16. A method of modulating site specific activity of a G-protein coupled receptor kinase (GRK) in a laboratory animal or an individual in need of enhanced or reduced signaling of a seven trans-membrane receptor comprising administering site specifically a GRK-derived HJ loop peptide, thereby modulating the site specific activity of the GRK.

17. A method of inhibiting site specific activity of a G-protein coupled receptor kinase (GRK) in an individual in need of enhanced signaling of a seven transmembrane receptor comprising administering, site specifically, a GRK-derived HJ loop peptide, thereby inhibiting the site specific activity of the GRK.

18. The method of claim 17 wherein the GRK is selected from a bARK1 or a bARK2 kinase family.

19. The method of claim 18 wherein the GRK is a bARK1 kinase.

20. The method of claim 17 wherein the GRK-derived HJ loop peptide is selected from the group cosisting of K024H001 (SEQ ID NO.:1), K024H003 (SEQ ID from the group cosisting of K024H001 (SEQ ID NO.:1), K024H003 (SEQ ID NO.:2), K024H007 (SEQ ID NO.:3), K024H101 (SEQ ID NO.:4), K024H102 (SEQ ID NO.:5), K024H103 (SEQ ID NO.:6), K024H104 (SEQ ID NO.:7), K024H105 (SEQ ID NO.:8), K024H106 (SEQ ID NO.:9), K024H107 (SEQ ID NO.:10), K024H108 (SEQ ID NO.:11), K024H109 (SEQ ID NO.:12), K024H110 (SEQ ID NO.:13), K024H111 (SEQ ID NO.:14), K024H112 (SEQ ID NO.:15), K024H113 (SEQ ID NO.:16), K024H114 (SEQ ID NO.:17), K024H901 (SEQ ID NO.:18), and K024H903 (SEQ ID NO.:19).

21. The method of claim 17 wherein the site specific activity of the GRK is melanogenesis.

22. A method of modulating activity of a G-protein coupled receptor kinase (GRK) in a cell culture comprising treating the cell culture with a GRK-derived HJ loop peptide, thereby inhibiting the activity of the GRK.

23. The method of claim 22 wherein the GRK is selected from a bARK1 or a bARK2 kinase family.

24. The method of claim 23 wherein the GRK is a bARK1 kinase.

25. The method of claim 22 wherein the GRK-derived HJ loop peptide is selected from the group cosisting of K024H001 (SEQ ID NO.:1), K024H003 (SEQ ID NO.:2), K024H007 (SEQ ID NO.:3), K024H101 (SEQ ID NO.:4), K024H102 (SEQ ID NO.:5), K024H103 (SEQ ID NO.:6), K024H104 (SEQ ID NO.:7), K024H105 (SEQ ID NO.:8), K024H106 (SEQ ID NO.:9), K024H107 (SEQ ID NO.:10), K024H108 (SEQ ID NO.:11), K024H109 (SEQ ID NO.:12), K024H110 (SEQ ID NO.:13), K024H111 (SEQ ID NO.:14), K024H112 (SEQ ID NO.:15), K024H113 (SEQ ID NO.:16), K024H114 (SEQ ID NO.:17), K024H901 (SEQ ID NO.:18), and K024H903 (SEQ ID NO.:19).

26. A method of treatment of Syndrome X or Type II diabetes mellitus in an individual in need thereof comprising administration of an inhibitor of GRK2 or GRK3 or said individual, wherein the administration results in reduction or elimination of Syndrome X symptoms or correction of Type II diabetes when compared to the Syndrome X symptoms or Type II diabetes indicia in the absence of said administration.

27. The method of claim 26 wherein the inhibitor is a GRK2-or GRK3-derived HJ loop peptide.

28. The method of claim 27 wherein the GRK2-or GRK-3 derived HJ loop peptide is selected from the group consisting of K024H001 (SEQ ID NO.:1), K024H003 (SEQ ID NO.:2), K024H007 (SEQ ID NO.:3), K024H101 (SEQ ID NO.:4), K024H102 (SEQ ID NO.:5), K024H103 (SEQ ID NO.:6), K024H104 (SEQ ID NO.:7), K024H105 (SEQ ID NO.:8), K024H106 (SEQ ID NO.:9), K024H107 (SEQ ID NO.:10), K024H108 (SEQ ID NO.:11), K024H109 (SEQ ID NO.:12), K024H110 (SEQ ID NO.:13), K024H111 (SEQ ID NO.:14), K024H112 (SEQ ID NO.:15), K024H113 (SEQ ID NO.:16), K024H114 (SEQ ID NO.:17), K024H901 (SEQ ID NO.:18), and K024H903 (SEQ ID NO.:19).

29. The method of claim 26 wherein said administering the GRK2- or GRK3-derived HJ loop peptide is systemic.

30. A peptide having an amino acid sequence selected from the group consisting of K024H001 (SEQ ID NO.:1), K024H003 (SEQ ID NO.:2), K024H007 (SEQ ID NO.:3), K024H101 (SEQ ID NO.:4), K024H102 (SEQ ID NO.:5), K024H103 (SEQ ID NO.:6), K024H104 (SEQ ID NO.:7), K024H105 (SEQ ID NO.:8), K024H106 (SEQ ID NO.:9), K024H107 (SEQ ID NO.:10), K024H108 (SEQ ID NO.:11), K024H109 (SEQ ID NO.:12), K024H110 (SEQ ID NO.:13), K024H111 (SEQ ID NO.:14), K024H112 (SEQ ID NO.:15), K024H113 (SEQ ID NO.:16), K024H114 (SEQ ID NO.:17), K024H901 (SEQ ID NO.:18), and K024H903 (SEQ ID NO.:19).
